(19) United States
(12) Patent Application Publication
(10) Pub. No.: US 2024/0390054 A1
(43) Pub. Date: Nov. 28, 2024

(54) CRYOPROBES AND RELATED METHODS

(71) Applicant: AtriCure, Inc., Mason, OH (US)

(72) Inventors: Justin David Volz, West Chester, OH (US); Jeffrey Arnold Beier, West Harrison, OH (US); Jared Edward Neidhard, Centerville, OH (US); Drew Douglas Schierloh, Glandort, OH (US); Jonathon McHale, Mason, OH (US); Craig Christipher Crandall, Rising Sun, IN (US); Stefan Stefanov, Ludlow, KY (US)

(73) Assignee: AtriCure, Inc., Mason, OH (US)

(21) Appl. No.: 18/670,897

(22) Filed: May 22, 2024

Related U.S. Application Data
(60) Provisional application No. 63/503,773, filed on May 23, 2023.

(51) Int. Cl. A61B 18/02; A61B 18/00

(52) U.S. Cl. CPC A61B 18/02; A61B 2018/00005; A61B 2018/00583; A61B 2018/0231

(57) ABSTRACT

A cryogenic probe, comprising: (a) a handle, (b) a shaft disposed distally on the handle, the shaft comprising a generally rigid proximal portion and a generally malleable distal portion, and (c) an active tip disposed distally on the shaft, where the shaft comprises a supply conduit configured to supply a cryogenic fluid to the active tip, an exhaust conduit configured to exhaust spent cryogenic fluid from the active tip, and a vacuum insulating layer disposed around the supply conduit and the exhaust conduit.

### CROSS-REFERENCE TO RELATED APPLICATIONS

The present application claims the benefit of U.S. Provisional Patent Application No. 63/503,773, filed on May 23, 2023 and titled “CRYOPROBES AND RELATED METHODS, the disclosure of which is hereby incorporated by reference in its entirety.

### INTRODUCTION TO THE INVENTION

The present disclosure is directed to cryogenic surgical devices, and, more specifically, to cryogenic surgical probes having a bendable shaft connected to an active tip, and related methods.

The present disclosure contemplates that cryogenic surgical devices, such as cryogenic probes, may be used in various medical and surgical procedures. Generally, cryogenic probes may be used to apply extremely cold temperatures to a target tissue. Cryogenic probes may be used for cryoablation and/or cryoanalgesia, for example.

The present disclosure contemplates that some cryogenic probes may be supplied with one or more cryogenic fluids, which may be used to cool a tissue-contacting, active portion, such as an ablation tip. Some cryogenic probes may include supply conduits, which convey cryogenic fluid to the ablation tip, and exhaust conduits, which convey used cryogenic fluid away from the ablation tip. Some cryogenic probes may utilize cryogenic fluids supplied at high pressures. For example, some cryogenic probes employing Joule-Thompson expansion at or near the ablation tip may receive liquid nitrous oxide at up to about 1200 psi and about room temperature and/or may exhaust the nitrous oxide as a gas or mixed phase of gas and liquid at about 45 psi and about −90° F. The cryogenic probe and associated conduits and connectors may be designed to withstand such pressures and temperatures.

The present disclosure contemplates that while cryoprobes may utilize extremely cold temperatures to achieve desired effects at target locations, exposure of other locations to extremely cold temperatures may cause undesired effects. For example, it may be desirable for the active tip of a cryoprobe to be extremely cold while the shaft on which the tip is disposed remains above a tissue-ablating temperature. The present disclosure provides methods and apparatus that improve the ability of cryoprobes to cool an active tip to a desired temperature while maintaining other external portions of the cryoprobe at warmer temperatures. In this manner, the present disclosure provides improvements over the prior art related to cryoprobes.

While known cryogenic devices have been used safely and effectively to perform cryosurgical procedures, improvements in the construction and operation of cryogenic probes may be beneficial for users (e.g., surgeons) and patients. The present disclosure includes various improvements that may enhance the construction, operation, and use of cryogenic probes.

It is a first aspect of the present invention to provide a cryogenic probe, comprising: (a) a handle, (b) a shaft disposed distally on the handle, the shaft comprising a generally rigid proximal portion and a generally malleable distal portion, and (c) an active tip disposed distally on the shaft, where the shaft comprises a supply conduit configured to supply a cryogenic fluid to the active tip, an exhaust conduit configured to exhaust spent cryogenic fluid from the active tip, and a vacuum insulating layer disposed around the supply conduit and the exhaust conduit.

In a more detailed embodiment of the first aspect, the shaft comprises a shell at least partially defining a radially outer aspect of the vacuum insulating layer, and the exhaust conduit at least partially defines a radially inner aspect of the vacuum insulating layer. In yet another more detailed embodiment, the supply conduit, the exhaust conduit, and the shell are concentrically disposed about a longitudinal axis of the shaft. In a further detailed embodiment, the cryogenic probe further includes a tubular thermal barrier element disposed within the vacuum insulating layer and radially between the exhaust conduit and the shell. In still a further detailed embodiment, the thermal barrier element is constructed of at least one of a ceramic, a foam, an elastomer, a composite, and fiberglass. In a more detailed embodiment, the cryoprobe further includes a distal end cap providing a sealed connection between the shell and the exhaust conduit, where the distal end cap comprises a scaling portion, the sealing portion comprising an internal bore configured to receive the exhaust conduit therein and at least one external circumferential surface configured to receive the shell. In a more detailed embodiment, the distal end cap is configured to engage the active tip, the active tip comprises a threaded portion, and the end cap comprises distal threads configured to threadedly engage the threaded portion of the active tip.

In yet another more detailed embodiment of the first aspect, the cryoprobe further includes a proximal end cap comprising an elongated proximal portion extending within the handle. In yet another more detailed embodiment, the cryoprobe further includes an adapter fluidically coupled to the proximal end cap, where the adapter converts a distal tube-in-tube arrangement of the supply conduit and exhaust conduit into a proximal side-by-side arrangement. In a further detailed embodiment, the cryoprobe further includes an outermost insulating cover disposed around the shell. In still a further detailed embodiment, the cryoprobe is accompanied by a cryosurgical module configured to at least one of supply the cryogenic fluid to the cryoprobe and receive the spent cryogenic fluid from the cryoprobe. In a more detailed embodiment, the shaft of the cryoprobe comprises at least one visible indicium configured to function as a visual aid during operation. In a more detailed embodiment, the at least one visible indicium comprises at least one circumferential band of contrasting color disposed on the shaft.

It is a second aspect of the present invention to provide a method of constructing a vacuum insulated tube, the method comprising: (a) applying braze paste to a mating surface of at least one of an inner tube, an outer tube, and an end cap, (b) engaging the end cap with the inner tube and the outer tube, (c) evacuating a volume between the inner tube and the outer tube, and (d) forming sealed braze joints between the end cap and the outer tube and between the end cap and the inner tube by heating at least one of the end cap, the outer tube, and the inner tube.

In a more detailed embodiment of the second aspect, the method further includes applying braze paste externally to an interface between the outer tube and the end cap. In yet another more detailed embodiment, the method further includes circumferentially crimping the outer tube into a circumferential groove of the end cap. In a further detailed embodiment, the inner tube includes a straight wall portion and a convoluted portion, and the braze joint between the end cap and the inner tube is formed along the straight wall portion. In still a further detailed embodiment, the outer tube includes a straight wall portion and a convoluted portion, and the braze joint between the end cap and the outer tube is formed along the straight wall portion. In a more detailed embodiment, the method further comprises positioning an internal insulator between the inner tube and the outer tube.

It is a third aspect of the present invention to provide a cryogenic probe, comprising: (a) a handle, (b) an insulated shaft disposed distally on the handle, and (c) an active tip disposed distally on the shaft, where the shaft comprises, from radially outside to radially inside, an outer covering, a shell, a thermal barrier, an exhaust conduit, and a supply conduit.

In yet another more detailed embodiment of the third aspect, the outer covering, the shell, the thermal barrier, the exhaust conduit, and the supply conduit are concentrically arranged. In yet another more detailed embodiment, the shell and the exhaust conduit at least partially define a vacuum insulating jacket comprising the thermal barrier. In a further detailed embodiment, the vacuum insulating jacket includes a thermal barrier material. In still a further detailed embodiment, the shaft comprises a generally rigid proximal portion and a generally malleable distal portion.

It is a fourth aspect of the present invention to provide a method of operating a cryosurgical probe, the method comprising: (a) bending a distal portion of a shaft of a cryosurgical probe into a desired configuration, (b) positioning an active tip of the cryosurgical probe in contact with a target tissue, and (c) supplying cryogenic fluid to the active tip via the shaft while maintaining a vacuum insulating jacket along the distal portion of the shaft.

In yet another more detailed embodiment of the fourth aspect, bending the distal portion of the shaft of the cryosurgical probe into the desired configuration comprises simultaneously bending a shell of the shaft, an exhaust conduit of the shaft, and a supply conduit of the shaft into the desired configuration. In yet another more detailed embodiment, bending the distal portion of the shaft of the cryosurgical probe into the desired configuration further comprises simultaneously bending a thermal barrier material disposed radially between the shell and the exhaust conduit within the vacuum insulating jacket. In a further detailed embodiment, the shell and the exhaust conduit are convoluted.

### BRIEF DESCRIPTION OF DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements may be exaggerated relative to other elements.

Embodiments incorporating teachings of the present disclosure are shown and described with respect to the figures presented herein.

FIG. 1 is an elevated perspective view of an exemplary cryogenic surgical system in accordance with the instant disclosure showing an exemplary cryoprobe with a bendable shaft capable of multiple shapes.

FIG. 2 is a longitudinal sectional view of the exemplary cryoprobe of FIG. 1 taken along line 2-2.

FIG. 3A is a profile view of an exemplary cryoprobe shaft in accordance with the instant disclosure.

FIG. 3B is a cross-sectional view of the exemplary cryoprobe shaft of FIG. 3A along line 3B-3B.

FIG. 4 is a cross-sectional view of the exemplary cryoprobe shaft of FIG. 3A along line 4-4.

FIG. 5 is a cross-sectional view of the exemplary distal end portion of the cryoprobe shaft of FIG. 3A.

FIG. 6A is an isometric view of a proximal end of an exemplary distal end cap in accordance with the instant disclosure.

FIG. 6B is an isometric view of a distal end of an exemplary distal end cap in accordance with the instant disclosure.

FIG. 7A is an elevated perspective view of the distal end cap of FIG. 6A being aligned with a distal end of an exhaust conduit and a shell to fabricate a vacuum insulated tube in accordance with the instant disclosure.

FIG. 7B is an elevated perspective view of the distal end cap of FIG. 6A being mounted to a distal end of an exhaust conduit and a shell to fabricate a vacuum insulated tube in accordance with the instant disclosure.

FIG. 8 is a magnified view of the handle and components housed therein as depicted in FIG. 2.

FIG. 9 is an elevated perspective view of an exemplary cryoprobe in accordance with the instant disclosure.

FIG. 10 is a profile view of an alternate exemplary cryoprobe shaft and distal tip in accordance with the instant disclosure.

### DETAILED DESCRIPTION

Example embodiments according to the present disclosure are described and illustrated below to encompass devices, methods, and techniques relating to cryogenic surgical devices, such as a cryogenic probe (“cryoprobe”) having a bendable shaft connected to an active tip, and related methods. Of course, it will be apparent to those of ordinary skill in the art that the embodiments discussed below are examples and may be reconfigured without departing from the scope and spirit of the present disclosure. It is also to be understood that variations of the example embodiments contemplated by one of ordinary skill in the art shall concurrently comprise part of the instant disclosure. However, for clarity and precision, the example embodiments as discussed below may include optional steps, methods, and features that one of ordinary skill should recognize as not being a requisite to fall within the scope of the present disclosure. Unless explicitly stated otherwise, any feature or function described in connection with any example embodiment may apply to any other example embodiment, and repeated description of similar features and functions is omitted for brevity.

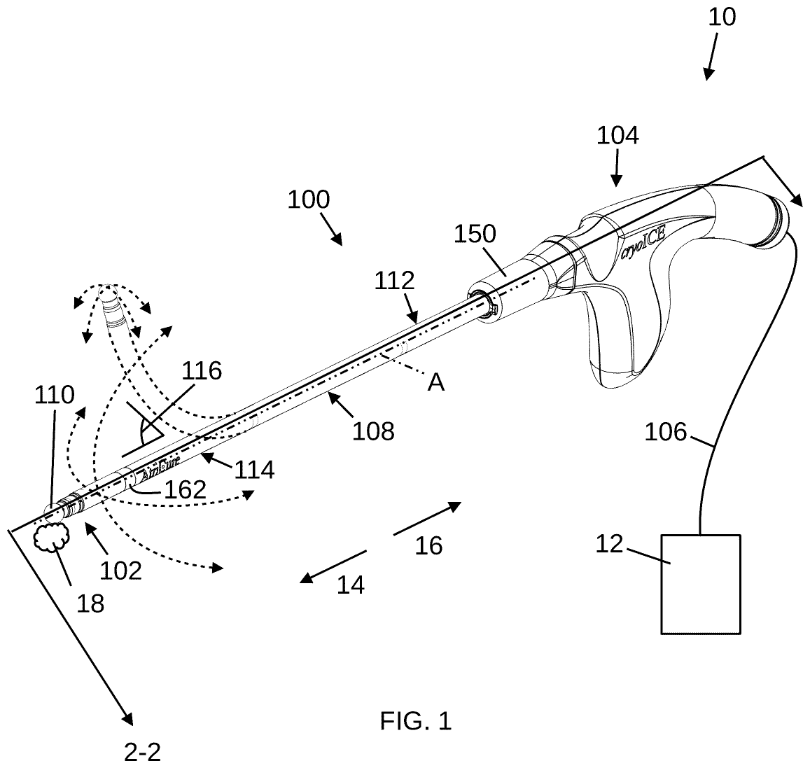

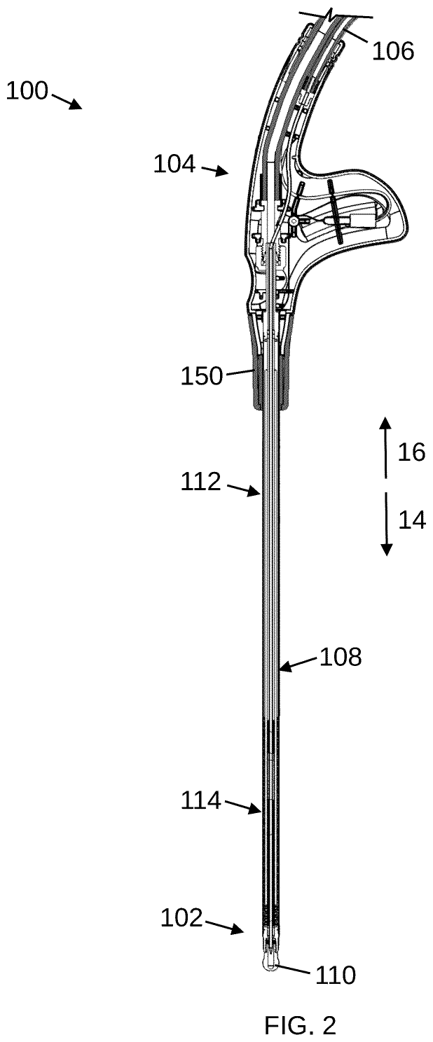

Referring to FIGS. 1 and 2, an exemplary cryoprobe 100 may be a part of a cryogenic surgical system 10, which may include a cryogenic module 12. The cryogenic probe 100 may be configured to operatively couple to the cryogenic module 12, which may be configured to supply cryogenic fluid to and/or receive cryogenic fluid from the cryogenic probe 100, as well as provide monitoring and/or control functions. The illustrative cryogenic module 12 may be generally similar to the “cryoICE BOX” cryogenic surgical unit available from AtriCure, Inc. of Mason, Ohio, or may include one or more components providing similar functionality. Example cryogenic fluids may include one or more of nitrous oxide, argon, carbon dioxide, and/or phase change fluids (e.g., liquid nitrogen). As used herein, “fluid” may refer to a substance in a liquid phase, a gas phase, a mixture of liquid and gas phases, and/or a supercritical phase.

The description herein references a distal direction 14 and a proximal direction 16. The proximal direction 16 may be generally opposite the distal direction 14. As used herein, “distal” may refer to a direction generally away from an operator of a system or device (e.g., a surgeon), such as toward the distant-most end of a device that is inserted into a patient's body. As used herein, “proximal” may refer to a direction generally toward an operator of a system or device (e.g., a surgeon), such as away from the distant-most end of a device that contacts a patient's body. It is to be understood, however, that example directions referenced herein are merely for purposes of explanation and clarity, and should not be considered limiting.

In the illustrated embodiment, the example cryogenic probe 100 may include a handle 104, which may be configured to be grasped by a user (e.g., surgeon) and/or engaged by a robotic device (e.g., a surgical robot). More generally, the handle 104 may comprise any structure that may be configured to be secured, held, and/or manipulated to position or restrain the cryogenic probe 100, regardless of whether it may be utilized by a human (e.g., surgeon or assistant), robot, mechanical device, etc. The handle 104 may be configured to connect to the cryogenic module 12 using one or more connecting elements 106, which may include one or more fluid conduits (e.g., for supplying and/or exhausting cryogenic fluid) and/or one or more electrical conductors (e.g., wiring for thermocouples), for example. The cryogenic probe 100 may include an elongated, generally tubular shaft 108 disposed generally distally on the handle 104. The distal end portion 102 of the cryogenic probe 100 may include an active tip 110, which may be disposed distally on the shaft 108. The active tip 110 may be utilized to apply extremely cold temperatures for cryogenic ablation of a target tissue 18, for example.

In some example embodiments, the shaft 108 may include a first, proximal portion 112 and/or a second, distal portion 114. In some example embodiments, the proximal portion 112 of the shaft 108 may be generally rigid and/or generally elastically deformable. As used herein, “rigid” may describe a shaft (or portion thereof) that does not deform or deforms minimally when subject to forces consisted with normal, intended use of the device. For example, the proximal portion 112 of the embodiment illustrated in FIG. 1 may be configured to retain and/or return to a generally straight shape.

In some example embodiments, the distal portion 114 of the shaft 108 may be bendable in one or more curves and/or in one or more planes. As used herein, “bendable” may describe a shaft (or portion thereof) that is elastically and/or plastically deformable in bending when subject to forces consistent with normal, intended use of the device. For example, a bendable shaft (or portion thereof) may be flexible and/or malleable. As used herein, “flexible” may describe a shaft (or portion thereof) that substantially deforms elastically and/or returns substantially to its original shape when an applied stress is removed, when subject to forces consistent with normal, intended use of the device. As used herein, “malleable” may describe a shaft (or portion thereof) that can be bent into a desired configuration and will remain substantially in that configuration when an applied stress is removed, when subject to forces consistent with normal, intended use of the device. Some example embodiments may be configured for bending by hand (e.g., without tools). Some example embodiments may be configured for bending using one or more tools, such as a manual, hand-held tubing bender tool.

In some example embodiments, the distal portion 112 and/or the proximal portion 114 of the shaft 108 may be configured to be more rigid than corresponding portions of similar devices. Accordingly, some such embodiments according to at least some aspects of the present disclosure may be capable of being utilized at more extreme angles associated with complex surgeries and/or more difficult entry angles as compared to similar devices.

In some example embodiments, the distal portion 114 of the shaft 108 may be configured for bending at an angle 116 relative to a longitudinal axis A of the shaft 108 and/or the proximal portion 112 of the shaft 108. For example, the distal portion 114 of the shaft 108 may be bendable about 180 degrees. As indicated by dashed lines with arrows in FIG. 1, the distal portion 114 of the shaft 108 may be bendable in any plane that is generally parallel to the longitudinal axis A. In some embodiments, the distal portion 114 of the shaft 108 may be bendable in more than one plane, as indicated by dashed lines with arrows in FIG. 1. For example, the distal portion 114 of the shaft 108 may be bendable both in a plane generally parallel to the longitudinal axis A of the shaft 108 (e.g., generally upward) as well as in a plane disposed obliquely relative to the longitudinal axis A of the shaft 108 (e.g., generally leftward). As used herein, “obliquely” may refer to an angle that is neither perpendicular nor parallel. In some such embodiments, the distal portion 114 of the shaft may be bent in a plane generally orthogonal to the longitudinal axis A of the shaft 108 and/or in a plane that is inclined relative to the longitudinal axis A of the shaft 108. In some embodiments, the distal portion 114 of the shaft 108 may be bendable in two or more curves in the same or different planes, such as upward and downward (or leftward and rightward), generally in an S-shape.

Although the example embodiment illustrated in FIG. 1 may include a shaft 108 including both a generally rigid portion 112 and a generally bendable portion 114, various alternative example embodiments may include shafts including only one or more generally bendable portions, only one or more generally rigid portions, or any combination of one or more generally bendable portions and/or one or more generally rigid portions, in any arrangement. In the illustrated embodiment, the generally rigid portion 112 is generally straight; however, alternative embodiments may include generally rigid portions 112 including one or more curves formed therein, such leftward, rightward, upward, and/or downward from the perspective of the user. In the illustrated embodiment, the generally rigid portion 112 is fixed relative to the handle so that the generally rigid portion is not rotatable relative to the handle 104. However, in an alternate exemplary embodiment, the generally rigid portion 112 may be rotatable relative to the handle 104.

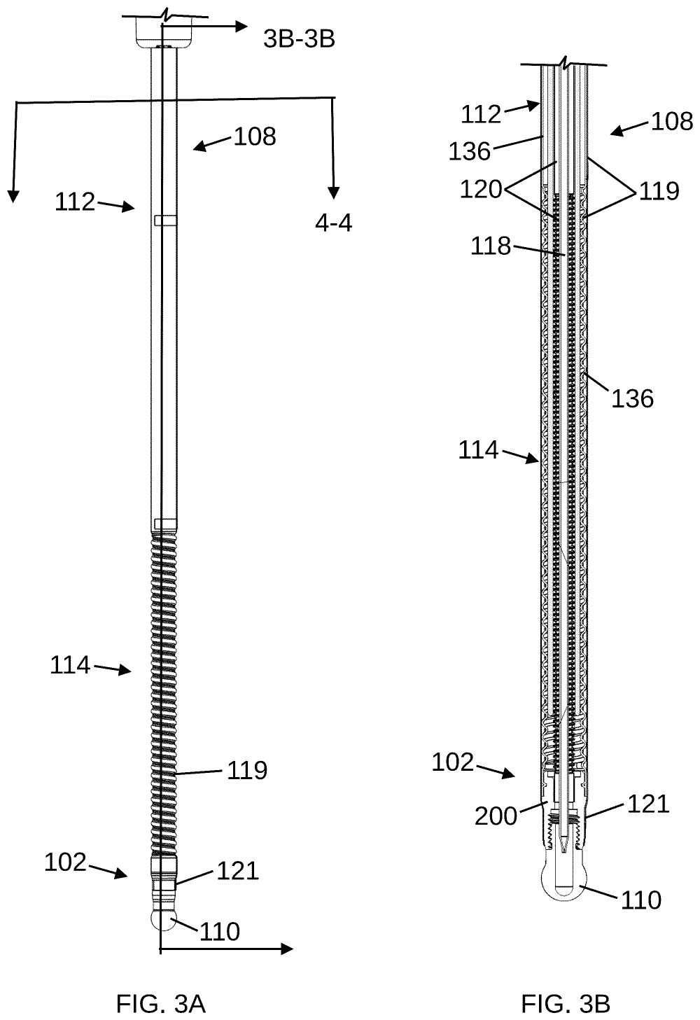

Turning to FIGS. 1-5, in some example embodiments, the shaft 108 may include one or more internal conduits (e.g., which may at least partially define corresponding lumens and/or flow paths) configured to direct cryogenic fluid to and/or from the handle 104 and/or one or more electrical conductors extending to and/or from the active tip 110 and/or the handle 104. For example, the shaft 108 may include a supply conduit 118 configured to convey cryogenic fluid from the handle 104 to the active tip 110, an exhaust conduit 120 configured to convey spent cryogenic fluid from the active tip 110, and/or one or more wires 119 connected to a thermocouple 121. In the illustrated embodiment, the supply conduit 118 is constructed from stainless steel (e.g., 304/304L stainless steel, temper: hard), and the exhaust conduit 120 is constructed from stainless steel, though alternative materials may be used in other embodiments. The thermocouple 121 may be configured for sensing a temperature during operation of the device, such as a temperature of the distal end portion 102 of the cryoprobe 100. In the illustrated embodiment, the thermocouple 121 may be positioned closer to the active tip 110 than in similar cryoprobes, thus providing more relevant temperature information than in other cryoprobes. As used herein, “spent cryogenic fluid” may refer to cryogenic fluid that has exited the active tip 110 (e.g., into the exhaust conduit 120), regardless of its phase, temperature, or pressure, and regardless of whether it may be capable of further cooling.

In the illustrated embodiment, the supply conduit 118 may be generally concentrically disposed within the exhaust conduit 120. As used herein, “concentric” may describe components which are arranged so that they have a common center point and/or axis (e.g., longitudinal axis A). In the illustrated embodiment, cryogenic fluid flowing to the active tip 110 flows within the supply conduit 118, and spent cryogenic fluid flowing from the active tip 110 flows through the annular lumen inside of the exhaust conduit 120 and outside of the supply conduit 118. The supply conduit 118 and/or the exhaust conduit 120 may be disposed generally concentrically within other components of the shaft 108. In alternative embodiments, the conduits 118, 120 may be disposed alongside one another (e.g., generally parallel), within one another non-concentrically, and/or within the shaft 108 non-concentrically.

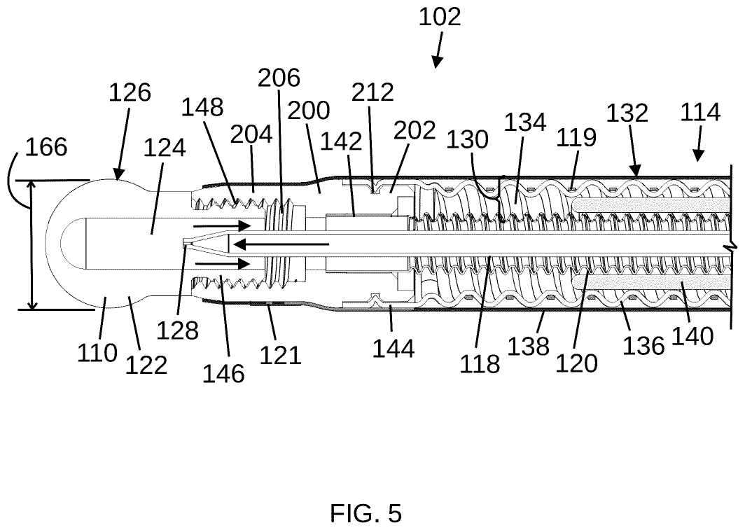

Referring to FIG. 5, in some example embodiments, the active tip 110 may include a wall 122 at least partially defining an internal cavity 124 with a closed distal end. In the illustrated embodiment, the active tip 110 may be constructed from aluminum, which has a relatively high thermal conductivity. In some alternative embodiments, the tip 110 may be constructed from other suitable materials, such as stainless steel. One skilled in the art will recognize that utilizing an alternative material with a different thermal conductivity may require changes to the design of some aspects of the tip 110, such as the wall 122 thickness, to achieve desired thermal performance. The wall 122 may at least partially define a generally rounded outer shape, or any other shape as desired for engagement with a target anatomy. For example, the active tip 110 may have a generally bulbous tissue contacting surface 126, which may have a diameter 166 of about 8 mm. As used herein, “bulbous” may refer to an enlarged (with respect to adjacent structure) and generally rounded exterior surface. Other exterior shapes and surfaces may be utilized for the active tip 110, such as, without limitation, a domed cylindrical surface, a rounded conical surface, or any of other various bulbous surfaces known to those skilled in the art.

In the illustrated embodiment, the active tip 110 may include a nozzle 128 (e.g., an orifice) through which cryogenic fluid from the supply conduit 118 enters the internal cavity 124. The internal cavity 124 may be fluidically coupled to the exhaust conduit 120. Thus, the internal cavity 124 of the active tip 110 may fluidically interpose the supply conduit 118 and the exhaust conduit 120.

In operation, cryogenic fluid supplied from the cryogenic module 12 may flow through the supply conduit 118 and the nozzle 128 into the internal cavity 124 of the active tip 110. Generally, the cryogenic fluid exiting the nozzle 128 may be at a higher pressure upstream from the nozzle 128 and may be allowed to expand downstream of the nozzle 128 within the cavity 124 at a significantly lower pressure, thereby creating a Joule-Thompson expansion and significantly lowering the temperature of the cryogenic fluid and the active tip 110. In some example embodiments utilizing nitrous oxide as a cryogenic fluid, the nitrous oxide may be supplied as a liquid at a temperature of about 27 C and a pressure of about 800 psi upstream of the nozzle 128, and may comprise a gaseous phase or a mixed phase of gas and liquid at approximately 45 psi and −68 C within the internal cavity 124 of the active tip 110. Alternatively, the cryogenic fluid may be supplied as a supercritical fluid. As Joule-Thompson expansion continues occurring within the active tip 110, the wall 122 becomes cooled enough for use in a cryosurgical procedure (e.g., cryoablation), such as by bringing the active tip 110 into contact with tissue to be ablated. By way of example, depending upon the cryogenic fluid utilized and the state of the fluid, exemplary flow rates for cryogenic fluid through the nozzle 128 may range between approximately fifteen to greater than one hundred cubic centimeters per minute.

The present disclosure contemplates that, during operation of the cryoprobe 100, cryogenic fluid supplied to the active tip 110 and/or cryogenic fluid exhausted from the active tip 110 may cause cooling of the shaft 108. In some circumstances, cryogenic fluid flowing through the shaft 108 may be cold enough to cause undesired thermal effects, such as freezing of non-target tissue or adherence of the shaft 108 to non-target tissue. For example, during procedures performed in the chest, it may be advantageous to avoid inadvertent freezing of or adherence to lung tissue and/or the periphery of an opening through the chest wall. Accordingly, in the illustrated embodiment, the shaft 108 includes an insulating portion 130 radially interposing the cryogenic fluid conduits 118, 120 and an outer surface 132 of the shaft 108. In some example embodiments, the shaft 108 may be configured to provide greater insulating capability than corresponding portions of similar devices, thus providing operational advantages.

In the illustrated embodiment, the insulating portion 130 may include a vacuum insulating layer 134 radially outward from the exhaust conduit 120. As used herein, “vacuum insulating layer” refers to an at least partially evacuated volume configured to reduce heat transfer, and includes a near vacuum, a partial vacuum, and a total vacuum. In the illustrated embodiment, the vacuum insulating layer 134 comprises a generally annular evacuated volume at least partially enclosed by an outer surface of the exhaust conduit 120 and an inner surface of a shell 136 of the shaft 108. In the illustrated embodiment, the shell 136 is constructed from stainless steel, and alternative materials may be used in other embodiments. In the illustrated embodiment, the shell 136 is at least partially covered by an outer covering 138, such as a heat shrink polymer tube disposed radially outward from the shell 136.

In the illustrated embodiment, a thermal barrier element 140 is disposed within the vacuum insulating layer 134, such as radially between the exhaust conduit 120 and the shell 136. For example, the thermal barrier element 140 may comprise a generally tubular, woven ceramic material. In alternative embodiments, the thermal barrier element 140 may comprise other insulative materials capable of withstanding both cryogenic temperatures and brazing temperature, such as fiberglass, elastomers, foams, silicon, carbon fiber, and composites. In some alternative embodiments without vacuum insulation, or in the illustrated embodiment if the integrity of the evacuated volume is compromised, the thermal barrier element 140 may be configured to provide thermal insulation sufficient to allow the device to meet desired performance requirements pertaining to shaft temperature.

In the illustrated embodiment, the shaft 108 is configured to provide the desired rigidity in the proximal portion 112 and the desired bendability in the distal portion 114 by utilizing a substantially straight-walled construction in the proximal portion 112 and a substantially convoluted construction in the distal portion 114. As used herein, “convoluted” refers to an object having a form or shape that is not smooth and may be folded in curved or tortuous windings, and includes corrugations, whether or not the corrugations are spiral or otherwise. In particular, in this embodiment, in the proximal portion 112, the supply conduit 118, the exhaust conduit 120, and the shell 136 are each formed as a substantially straight walled (e.g., non-convoluted) tube. In the distal portion 114, the exhaust conduit 120 and the shell 136 are each formed as a substantially convoluted tube. In the illustrated embodiment, the convolutions are spiraled and/or helical in shape; however, other convolutions such as circumferential corrugations may be utilized in alternative embodiments. In some example embodiments, one or more wires 119 connected to the thermocouple 121 may be routed along the distal convoluted portion 114 of the shaft 108 in a generally helical manner, such as wrapped around the convoluted portion 114 within the generally helical grooves. In some alternative embodiments, the supply conduit 118, the exhaust conduit 120, and/or the shell 136 may include convolutions in the generally rigid, proximal portion 112 of the shaft 108.

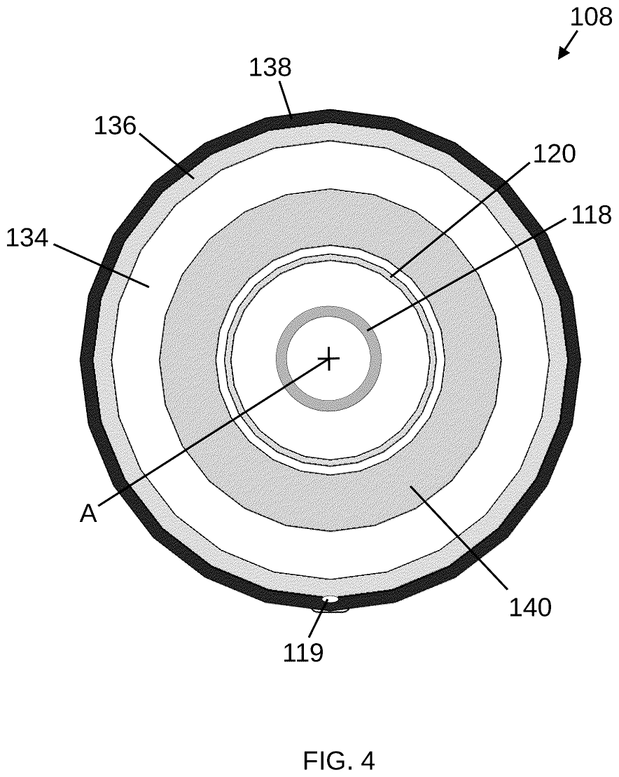

Referring to FIG. 4, in the illustrated embodiment, the proximal portion 112 of the shaft 108 may include one or more of the following components, in the following order, from outside to inside: outer covering 138, shell 136 (e.g., straight wall), thermal barrier 140, exhaust conduit 120 (e.g., straight wall), and/or supply conduit 118 (e.g., straight wall).

Referencing FIG. 5, in the illustrated embodiment, the distal portion 114 of the shaft 108 may include one or more of the following components, in the following order, from outside to inside: outer covering 138, shell 136 (e.g., convoluted), thermal barrier 140, exhaust conduit 120 (e.g., convoluted), and/or supply conduit 118 (e.g., straight wall).

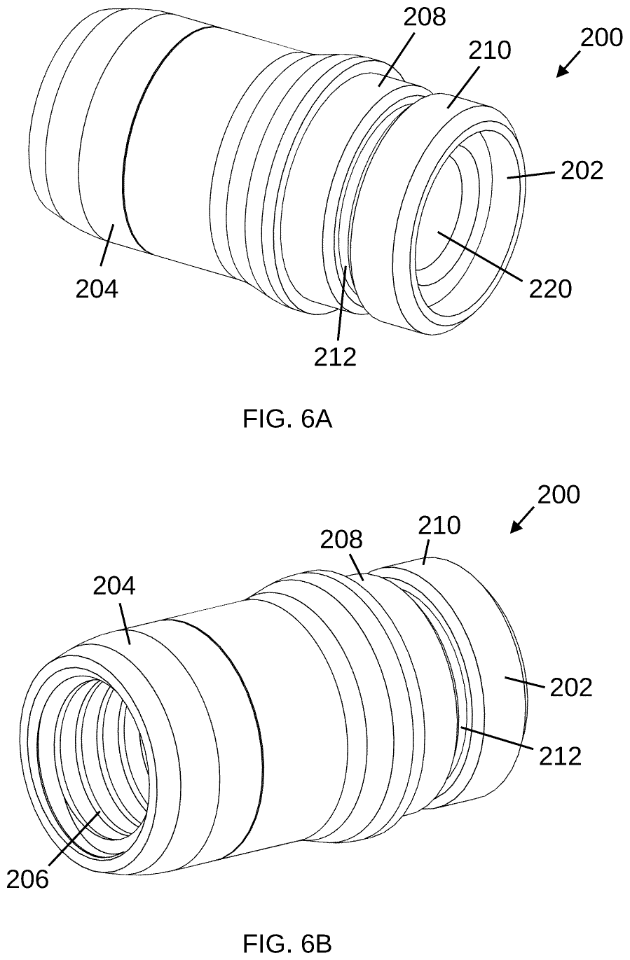

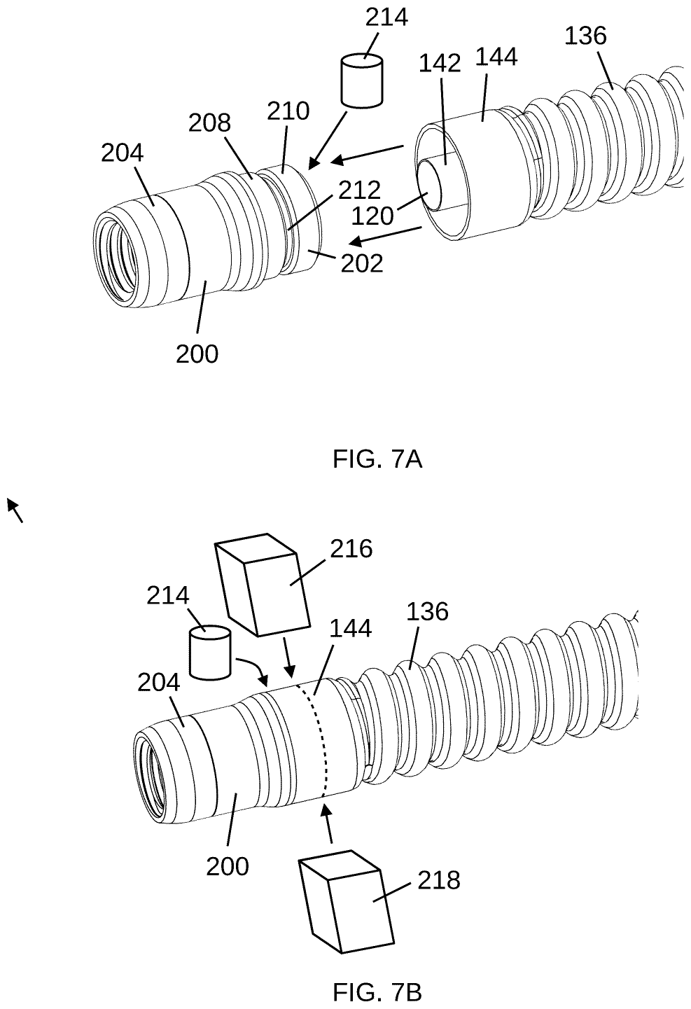

Referring to FIGS. 5, 6A, 6B, 7A, and 7B, in the illustrated embodiment, a distal end portion 142 of the exhaust conduit 120 (which may be straight walled) and a distal end portion 144 of the shell 136 (which may be straight walled) are sealed to a proximal sealing portion 202 of a distal end cap 200 to at least partially seal off the vacuum insulating layer 134. A distal end portion 204 of the distal end cap 200 includes an internally threaded recess 206 configured to engage an externally threaded proximal end portion 146 of the active tip 110.

In the illustrated embodiment, the distal end cap 200 is constructed from stainless steel. In some embodiments, the threaded connection between the threaded proximal end portion 146 of the active tip 110 and the internally threaded recess 206 of the distal end cap 200 may be sealed with epoxy 148. This may be particularly useful when the active tip 110 and the distal end cap 200 are constructed if different materials (e.g., aluminum and stainless steel, respectively). In some alternative embodiments, suitable alternative thread sealants may be used in place of the epoxy 148 (e.g., thread seal tape (e.g., PTFE), biocompatible room-temperature-vulcanizing silicone elastomers, etc.). In some alternative embodiments, joints may be sealed using suitable gaskets and/or O-rings, such as those constructed from materials capable of withstanding the low temperatures associated with cryosurgery. When the distal end cap 200 and the active tip 110 are constructed of the same material, or compatible materials, other joining methods, such as welding, brazing, etc. may be used.

In the illustrated embodiment, the proximal sealing portion 202 of the distal end cap 200 includes an internal bore 220 configured to receive the distal end portion 142 of the exhaust conduit 120 therein in a sealed manner. The proximal sealing portion 202 of the distal end cap 200 is configured to be received within the distal end portion 144 of the shell 136 in a sealed manner. For example, the distal end portion 142 of the exhaust conduit 120 and/or the distal end portion 144 of the shell 136 may be sealed to the proximal sealing portion 202 of the distal end cap 200 by respective braze joints.

In the illustrated embodiment, the proximal sealing portion 202 of the distal end cap 200 includes two external circumferential surfaces 208, 210 interposed by a circumferential groove 212. The external circumferential surfaces 208, 210 are configured to engage the radially inner surface of the distal end portion 144 of the shell 136. The groove 212 may be configured to engage the shell 136, such as when the shell 136 is crimped to the proximal sealing portion 202 of the distal end cap 200.

Referring again to FIGS. 5, 6A, 6B, 7A, and 7B, an example method of making a vacuum insulated tube, such as for a cryoprobe, may include obtaining an inner tube (e.g., the exhaust conduit 120), an outer tube (e.g., the shell 136), an end cap (e.g., the distal end cap 200), and/or an internal insulator (e.g., thermal barrier 140). Obtaining the inner tube 120 may include forming a tube having a straight walled portion and a convoluted portion and/or obtaining the outer tube 136 may include forming a tube having a straight walled portion and a convoluted portion. The method may include positioning the internal insulator 140 between the inner tube 120 and the outer tube 136. For example, the internal insulator 140 may be disposed on the inner tube 120, then the inner tube 120 and internal insulator 140 may be positioned within the outer tube 136. The method may include applying braze paste 214 in connection with forming brazed joints between the end cap 200 and one or more of the tubes 120, 136. For example, braze paste 214 may be applied to internal bore 220 and/or to other the mating surfaces of the inner tube 120, the outer tube 136, and/or the end cap 200. The method may include engaging the end cap 200 with the inner tube 120 and/or the outer tube 136. In the illustrated embodiment, the method may include crimping the outer tube 136 onto the end cap 200, such as by using one or more crimping tools 216, 218. For example, the outer tube 136 may be crimped generally circumferentially into the circumferential groove 212 of the end cap 200. In some embodiments, braze paste 214 may be applied externally, such as at the interface between the distal end of the outer tube 136 and the end cap 200. Although the exemplary method has been described in connection with the distal components of the insulated tube, it will be understood that similar operations may be performed to install a proximal end cap at the other (proximal) end of the insulated tube. The method may include heating the assembly to form the braze joints, such as in a vacuum furnace. Before and/or during the heating, a vacuum may be drawn in the furnace, which may evacuate the evacuated volume comprising the vacuum insulating layer 134.

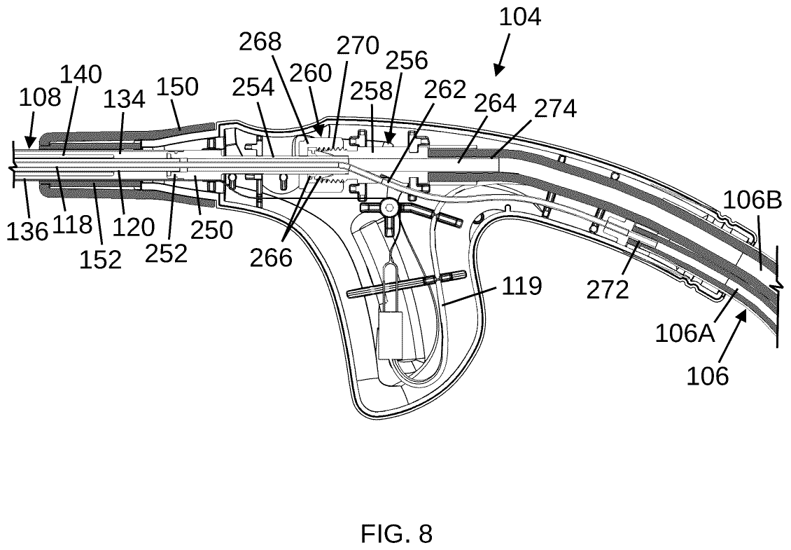

Referencing FIGS. 5, 6A, 6B, 7A, 7B, and 8, the proximal end of the vacuum insulating layer 134 may be sealed with a proximal end cap 250, which may be generally similar to the distal end cap 200. For example, the proximal end cap 250 may include a distal scaling portion 252 having features corresponding to those of the proximal sealing portion 202 of the distal end cap 200. Similarly, the proximal end cap 250 may be disposed on the exhaust conduit 120 and shell 136 in a manner generally similar to that described with reference to the distal end cap 200.

In the illustrated embodiment, the proximal end cap 250 includes an elongated proximal portion 254, which may extend proximally into sealed engagement with an adapter assembly 256. The adapter assembly 256 may be configured to convert the tube-in-tube (e.g., concentric) arrangement of the supply conduit 118 and exhaust conduit 120 of the shaft 108 to the side-by-side (e.g., parallel) arrangement of a supply connecting element 106A and an exhaust connecting element 106B comprising the connecting element 106.

In the illustrated embodiment, the adapter assembly 256 comprises an adapter body 258, a compression fitting 260 disposed distally on the adapter body 258 and configured for sealed engagement with the elongated proximal portion 254 of the proximal end cap 250, a supply interface 262, and an exhaust interface 264. The compression fitting 260 comprises a two-piece ferrule 266 (e.g., a cooperating front ferrule and back ferrule) disposed around the elongated proximal portion 254 of the proximal end cap 250 and an internally threaded nut 268 configured for threaded engagement with distal, external threads 270 of the adapter body 258. Tightening the nut 268 onto the adapter body 258 causes the ferrule 266 to create a sealed connection between the elongated proximal portion 254 of the proximal end cap 250 and the adapter body 258.

In the illustrated embodiment, the supply interface 262 comprises a proximally disposed extension of the supply conduit 118 that extends through, and seals with, the adapter body 258. In the illustrated embodiment, the supply conduit 118 is sealed to the adapter body 258 by a brazed connection. In some example embodiments, the supply conduit 118 may be spot welded to the adapter body 258, such as before a brazing operation. The proximal end of the supply interface 262 includes a barb fitting 272 (or other suitable fitting) configured for sealed engagement with the supply connecting element 106A (e.g., a hose), such as by using a crimp sleeve. In the illustrated embodiment, the exhaust interface 264 comprises a proximally disposed barb fitting 274 (or other suitable fitting) configured for sealed engagement with the exhaust connecting element 106B (e.g., a hose), such as by using a crimp sleeve. Thermocouple wires 119 (or extensions thereof) may run through the handle 104 to the connecting elements 106.

Referring to FIGS. 1, 2, and 8, some example embodiments may include one or more additional thermal barriers, such as at locations where patient or operator contacting surfaces may be subject to undesirably cool temperatures. For example, under some operating conditions, areas near the distal aspect of the handle 104 (around the proximal end of the shaft 108) may be cooled to temperatures that may be uncomfortable for an operator for extended periods of time. Accordingly, in the illustrated embodiment, the handle 104 includes a distally extending nose guard 150, generally in the form of a cone, disposed around the proximal aspect of the shaft 108. In some embodiments, an additional thermal barrier 152, which may be generally in the form of an annular cylinder, may be disposed around the shaft 108 and/or within the nose cone 150. In some example embodiments, part or all of the nose cone 150 may be constructed with an external color (or other indicium) that is visibly distinct from the handle 104, thus indicating to operators that they should limit contact with the nose cone 150.

Referring to FIGS. 1, 8, and 9, in the illustrated embodiment, the cryoprobe 100 includes various proximally disposed connectors configured to releasably attach the connecting elements 106 to the cryogenic module 12. For example, the cryoprobe 100 may include a supply connector 154 (which may be configured to attach the supply connecting element 106A to the cryogenic module 12), an exhaust connector 156 (which may be configured to attached the exhaust connecting element 106B to the cryogenic module 12), and/or one more thermocouple connectors 158, 160 (which may be configured to attach one or more thermocouple wires 119 to the cryogenic module 12).

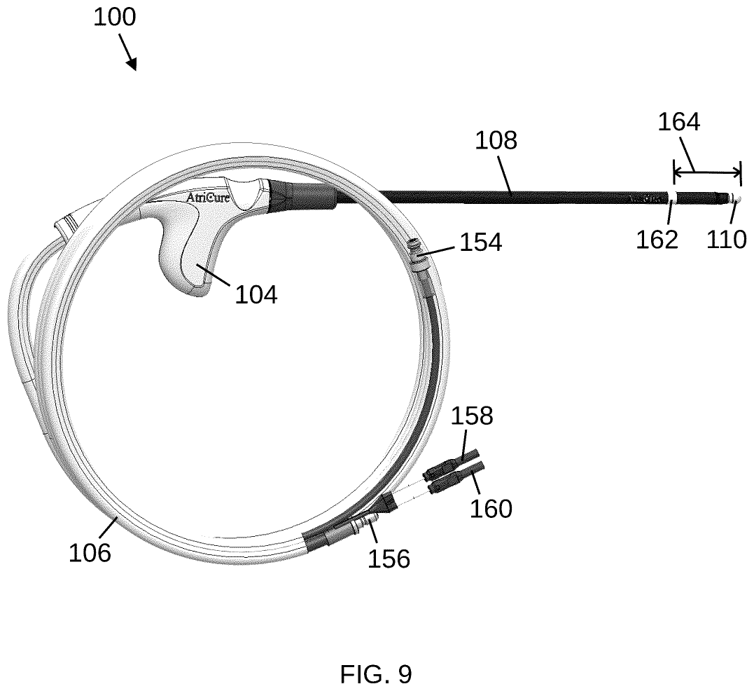

Referring to FIGS. 1 and 9, the shaft 108 may include at least one visible indicium configured to act as a visual aid for the operator. For example, the illustrated embodiment includes a visually contrasting, circumferential band 162 disposed on the shaft 108 at a longitudinal distance 164 from the distal tip of the active tip 110. In some embodiments, the distance 164 may be about 4 cm. Such visual indicia may be used by an operator as a reference for positioning the active tip 110 relative to visible anatomic structures and/or to avoid unintended cooling of nearby tissues.

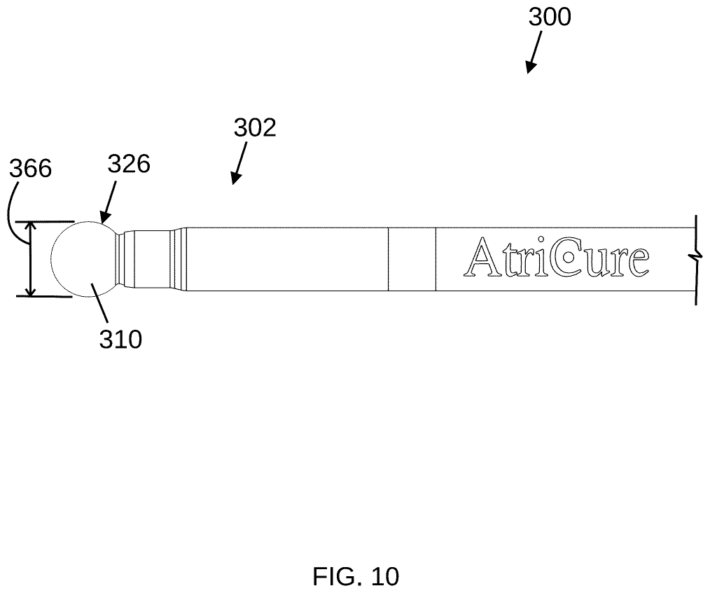

Turning to FIG. 10, an alternative example cryoprobe 300 may be generally similar to cryoprobe 100, may include similar components, and may be constructed and utilized in similar manners. Repeated description of corresponding structures and operations is omitted for brevity. In the cryoprobe 300, a distal portion 302 may include an active tip 310. The active tip 310 may have a generally bulbous tissue contacting surface 326. Referring to FIGS. 5 and 10, the active tip 110 of the cryoprobe 100 may have a diameter 166 of about 8 mm, while the active tip 310 of the cryoprobe 300 may have a diameter 366 of about 10 mm. Other alternative embodiments may include active tips with other suitable diameters and bulbous shapes.

Example methods of operating a cryoprobe 100, 300 may include bending the distal portion 114, 302 of the shaft 108 into a desired configuration, connecting the cryoprobe 100, 300 to a cryogenic module 12, positioning the active tip 110, 310 in contact with a target tissue 18, and/or supplying cryogenic fluid to and/or exhausting cryogenic fluid from the active tip 110, 310 to cool the active tip 110, 310. The bending operation may include simultaneously bending one or more of the components of the shaft (e.g., outer covering 138, shell 136, thermal barrier 140, exhaust conduit 120, and/or supply conduit 118). Further operations may include defrosting the active tip 110, 310 and/or repeating any of the above operations, at the same or different locations.

The following patent references may provide context for the present disclosure and are incorporated by reference herein in their entireties: U.S. Pat. No. 8,915,908, issued Dec. 23, 2014; U.S. Patent Application Publication No. 2020/0085485, published Mar. 19, 2020; U.S. Pat. No. 11,179,185, issued Nov. 23, 2021; U.S. Patent Application Publication No. 2023/0061212, published Mar. 2, 2023; U.S. Patent Application Publication No. 2023/0067890, published Mar. 2, 2023; and U.S. Patent Application Publication No. 2023/0063557, published Mar. 2, 2023. Generally, any features or improvements described herein may be used in connection with exemplary embodiments described in these patent references, and any features, elements, or methods described in these patent references may be used in connection with any of the embodiments described herein.

Following from the above description and invention summaries, it should be apparent to those of ordinary skill in the art that, while the methods and apparatuses herein described constitute example embodiments according to the present disclosure, it is to be understood that the scope of the disclosure contained herein is not limited to the above precise embodiments and that changes may be made without departing from the scope as defined by the following claims. Likewise, it is to be understood that it is not necessary to meet any or all of the identified advantages or objects disclosed herein in order to fall within the scope of the claims, since inherent and/or unforeseen advantages may exist even though they may not have been explicitly discussed herein.

What is claimed is:

1. A cryogenic probe, comprising:
a handle;
a shaft disposed distally on the handle, the shaft comprising a generally rigid proximal portion and a generally malleable distal portion; and
an active tip disposed distally on the shaft;
wherein the shaft comprises a supply conduit configured to supply a cryogenic fluid to the active tip, an exhaust conduit configured to exhaust spent cryogenic fluid from the active tip, and a vacuum insulating layer disposed around the supply conduit and the exhaust conduit.

2. The cryogenic probe of claim 1,
wherein the shaft comprises a shell at least partially defining a radially outer aspect of the vacuum insulating layer;
wherein the exhaust conduit at least partially defines a radially inner aspect of the vacuum insulating layer.

3. The cryogenic probe of claim 2, wherein the supply conduit, the exhaust conduit, and the shell are concentrically disposed about a longitudinal axis of the shaft.

4. The cryogenic probe of claim 2, further comprising a tubular thermal barrier element disposed within the vacuum insulating layer and radially between the exhaust conduit and the shell.

5. (cancelled)

6. The cryogenic probe of claim 2,
further comprising a distal end cap providing a sealed connection between the shell and the exhaust conduit;
wherein the distal end cap comprises a sealing portion, the sealing portion comprising an internal bore configured to receive the exhaust conduit therein and at least one external circumferential surface configured to receive the shell.

7. (canceled)

8. (canceled)

9. The cryogenic probe of claim 2, further comprising a proximal end cap comprising an elongated proximal portion extending within the handle.

10. (canceled)

11. The cryogenic probe of claim 2, further comprising an outermost insulating cover disposed around the shell.

12. A cryogenic surgical system, comprising
the cryoprobe of claim 1; and
a cryosurgical module configured to at least one of supply the cryogenic fluid to the cryoprobe and receive the spent cryogenic fluid from the cryoprobe.

13. The cryogenic probe of claim 1, wherein the shaft comprises at least one visible indicium configured to function as a visual aid during operation.

14. The cryogenic probe of claim 1, wherein the at least one visible indicium comprises at least one circumferential band of contrasting color disposed on the shaft.

15. A method of constructing a vacuum insulated tube, the method comprising:
applying braze paste to a mating surface of at least one of an inner tube, an outer tube, and an end cap;
engaging the end cap with the inner tube and the outer tube;
evacuating a volume between the inner tube and the outer tube; and
forming sealed braze joints between the end cap and the outer tube and between the end cap and the inner tube by heating at least one of the end cap, the outer tube, and the inner tube.

16. The method of claim 15, further comprising applying braze paste externally to an interface between the outer tube and the end cap.

17. The method of claim 15, further comprising circumferentially crimping the outer tube into a circumferential groove of the end cap.

18. The method of claim 15, wherein:
the inner tube includes a straight wall portion and a convoluted portion; and
the braze joint between the end cap and the inner tube is formed along the straight wall portion.

19. The method of claim 15, wherein:
the outer tube includes a straight wall portion and a convoluted portion; and
the braze joint between the end cap and the outer tube is formed along the straight wall portion.

20. (canceled)

21. A cryogenic probe, comprising
a handle;
an insulated shaft disposed distally on the handle; and
an active tip disposed distally on the shaft;
wherein the shaft comprises, from radially outside to radially inside, an outer covering, a shell, a thermal barrier, an exhaust conduit, and a supply conduit.

22. The cryogenic probe of claim 21, wherein the outer covering, the shell, the thermal barrier, the exhaust conduit, and the supply conduit are concentrically arranged.

23. The cryogenic probe of claim 21, wherein the shell and the exhaust conduit at least partially define a vacuum insulating jacket comprising the thermal barrier.

24. (canceled)

25. (canceled)

26. A method of operating a cryosurgical probe, the method comprising:
bending a distal portion of a shaft of a cryosurgical probe into a desired configuration;
positioning an active tip of the cryosurgical probe in contact with a target tissue; and
supplying cryogenic fluid to the active tip via the shaft while maintaining a vacuum insulating jacket along the distal portion of the shaft.

27. The method of claim 26, wherein bending the distal portion of the shaft of the cryosurgical probe into the desired configuration comprises simultaneously bending a shell of the shaft, an exhaust conduit of the shaft, and a supply conduit of the shaft into the desired configuration.

28. (canceled)

29. (canceled)
